(19) United States
(12) Patent Application Publication
(10) Pub. No.: US 2026/0183205 A1
(43) Pub. Date: Jul. 2, 2026

(54) OIL-IN-WATER TYPE SOLID COSMETIC MATERIAL AND COSMETIC PRODUCT

(71) Applicant: TOKIWA CORPORATION, Gifu (JP)

(72) Inventor: Shaolong LIN, Saitama (JP)

(73) Assignee: TOKIWA CORPORATION, Gifu (JP)

(21) Appl. No.: 19/428,907

(22) Filed: Dec. 22, 2025

(30) Foreign Application Priority Data
Dec. 26, 2024 (JP) 2024-229779

(51) Int. Cl. A61K 8/06; A61K 8/02; A61K 8/34; A61K 8/39; A61K 8/73; A61K 8/81; A61K 8/894; A61K 8/92; A61Q 1/08; A61Q 1/10; A61Q 19/00

(52) U.S. Cl. CPC A61K 8/062; A61K 8/0229; A61K 8/342; A61K 8/39; A61K 8/73; A61K 8/8158; A61K 8/894; A61K 8/922; A61Q 19/00; A61K 2800/594; A61K 2800/87; A61Q 1/08; A61Q 1/10

(57) ABSTRACT

An oil-in-water type solid cosmetic material includes (A) an aliphatic alcohol having a melting point of 40° C. or higher, (B) at least one kind of polyglycerin derivative surfactant selected from the group consisting of a polyglycerin fatty acid ester and a polyglycerin-modified silicone, (C) a gellan gum, and (D) a water-soluble thickener, in which a content of the component (C) is 0.22% to 1.3% by mass with respect to a total amount of the oil-in-water type solid cosmetic material.

### TECHNICAL FIELD

The present disclosure relates to an oil-in-water type solid cosmetic material and a cosmetic product.

### BACKGROUND

Makeup cosmetic materials such as lipsticks and foundations, which are stored in stick containers and used, are often applied by directly contacting a molded stick-shaped body with the skin of the lips or the like. For these applications, oily solid cosmetic materials having excellent moldability are generally used. The oily solid cosmetic materials are configured to include an oily component such as a wax and a liquid oil, and a powder component such as a pigment, and the composition is adjusted not only to provide moldability, but also to achieve shape retention that resists deformation during use, and usability such as ease of spreading on the skin and good adhesiveness to the skin. However, the oily solid cosmetic materials have a tackiness due to the oily component and a heavy sensory feel due to the wax.

As cosmetic materials that are applied directly to the skin, cosmetic materials including an aqueous component that provides a pleasant sensory feel to the skin, such as freshness and a cooling sensation, have attracted attention. For example, Japanese Unexamined Patent Publication No. H03-279319 proposes a solid-like oil-in-water type makeup cosmetic material containing water, a fatty acid soap, an oily component, and powder. In addition, Japanese Unexamined Patent Publication No. 2023-88747 proposes an oil-in-water type solid cosmetic material containing sulfur-containing polysaccharides, xyloglucan, and wax.

### SUMMARY

However, oil-in-water type solid cosmetic materials in the related art, in a case of being molded into a stick shape and applied directly to the skin, do not sufficiently exhibit shape retention and usability as obtained with oily solid cosmetic materials, and there is still room for improvement.

The present invention has been made in view of the circumstances, and an object of the present invention is to provide an oil-in-water type solid cosmetic material, which can be molded into a desired shape, has sufficient shape retention and usability such as ease of spreading on the skin and good adhesiveness to the skin, and can provide a pleasant sensory feel on the skin, such as freshness and a cooling sensation; and a cosmetic product including the same.

In order to solve the problem, the present invention provides the following inventions.
[1] An oil-in-water type solid cosmetic material including:
(A) an aliphatic alcohol having a melting point of 40° C. or higher;
(B) at least one kind of polyglycerin derivative surfactant selected from the group consisting of a polyglycerin fatty acid ester and a polyglycerin-modified silicone;
(C) a gellan gum; and
(D) a water-soluble thickener,
in which a content of the component (C) is 0.22% to 1.3% by mass with respect to a total amount of the oil-in-water type solid cosmetic material.
[2] The oil-in-water type solid cosmetic material according to [1], in which a total content of water-soluble solidifying agents (including the component (C)) included in the oil-in-water type solid cosmetic material is 1% by mass or less with respect to the total amount of the oil-in-water type solid cosmetic material.
[3] The oil-in-water type solid cosmetic material according to [1] or [2], in which a total content of solid oils included in the oil-in-water type solid cosmetic material is 7% to 20% by mass with respect to the total amount of the oil-in-water type solid cosmetic material.
[4] The oil-in-water type solid cosmetic material according to any one of [1] to [3], in which a mass ratio C/D of the content of the component (C) to a content of the component (D) is 0.3 to 5.
[5] The oil-in-water type solid cosmetic material according to any one of [1] to [4], in which the component (D) includes a polyacrylamide compound including an acryloyl dimethyltaurine salt as a constitutional unit.
[6] The oil-in-water type solid cosmetic material according to any one of [1] to [5], further including: a plant-derived wax.
[7] The oil-in-water type solid cosmetic material according to any one of [1] to [6], in which a content of water in the oil-in-water type solid cosmetic material is 20% to 70% by mass with respect to the total amount of the oil-in-water type solid cosmetic material.
[8] The oil-in-water type solid cosmetic material according to any one of [1] to [7], in which the oil-in-water type solid cosmetic material has a hardness of 0.7 to 1.5 N.
[9] The oil-in-water type solid cosmetic material according to any one of [1] to [8], in which the component (A) is an alcohol having a linear saturated hydrocarbon group having 18 to 22 carbon atoms.
[10] The oil-in-water type solid cosmetic material according to any one of [1] to [9], in which the oil-in-water type solid cosmetic material includes at least one selected from the group consisting of polyglyceryl-10 diisostearate and polyglyceryl-3 disiloxane dimethicone as the component (B).
[11] The oil-in-water type solid cosmetic material according to any one of [1] to [10],
   in which a total content of solid oils included in the oil-in-water type solid cosmetic material is 7% to 20% by mass with respect to the total amount of the oil-in-water type solid cosmetic material,
   a content of the component (A) in the oil-in-water type solid cosmetic material is 6% to 12% by mass with respect to the total amount of the oil-in-water type solid cosmetic material,
   a content of the component (B) in the oil-in-water type solid cosmetic material is 1% to 3% by mass with respect to the total amount of the oil-in-water type solid cosmetic material,
   a mass ratio C/D of the content of the component (C) to a content of the component (D) is 0.3 to 5, and
   a content of water in the oil-in-water type solid cosmetic material is 20% to 70% by mass with respect to the total amount of the oil-in-water type solid cosmetic material.
[12] A cosmetic product including:
   a container; and
   the oil-in-water type solid cosmetic material according to any one of [1] to [11] stored in the container,
   in which the container has
   a container main body holding the oil-in-water type solid cosmetic material, the oil-in-water type solid cosmetic material being held in the container main body in a state of protruding from the container main body, or
   a container main body accommodating the oil-in-water type solid cosmetic material, and having a holding body capable of causing the oil-in-water type solid cosmetic material to protrude from the container main body while holding the oil-in-water type solid cosmetic material, and
   the oil-in-water type solid cosmetic material is applied by direct contact with skin.
[13] The cosmetic product according to [12], in which the oil-in-water type solid cosmetic material has a stick shape.

The oil-in-water type solid cosmetic material according to [1], by adopting the configuration, can be molded into a desired shape (for example, a stick shape), and has sufficient shape retention and usability such as ease of spreading on the skin and good adhesiveness to the skin, and can provide a pleasant sensory feel on the skin, such as freshness and a cooling sensation. The present inventors speculate the following as reasons why such effects are achieved. The moldability and the shape retention of the cosmetic material including an aqueous component are also improved by increasing the content of the water-soluble solidifying agent, but in this case, it is difficult to obtain ease of spreading to the skin and adhesiveness. On the other hand, in the present invention, it is considered that the use of a specific amount of a specific water-soluble solidifying agent (component (C)) in combination with the component (A) and the component (B) makes it possible to sufficiently solidify the oil-in-water type cosmetic material and to form a molded body having sufficient shape retention. Furthermore, it is speculated that the use of the component (D) in combination makes it possible to obtain a molded body having usability such as ease of spreading to the skin and good adhesiveness (for example, a sufficient coloring effect can be obtained) without making the molded body too hard.

According to the present invention, provided are an oil-in-water type solid cosmetic material that can be molded into a desired shape, has sufficient shape retention and usability such as ease of spreading on the skin and good adhesiveness to the skin, and can provide a pleasant sensory feel on the skin, such as freshness and a cooling sensation, and a cosmetic product including the same.

### BRIEF DESCRIPTION OF DRAWING

FIG. 1 is a schematic cross-sectional view showing an embodiment of a cosmetic product.

### DETAILED DESCRIPTION

The oil-in-water type solid cosmetic material of the present embodiment (which is hereinafter also simply referred to as a “cosmetic material”) includes (A) an aliphatic alcohol having a melting point of 40° C. or higher (which may also be referred to as a “component (A)”), (B) at least one kind of polyglycerin derivative surfactant selected from the group consisting of a polyglycerin fatty acid ester and a polyglycerin-modified silicone (which may also be referred to as a “component (B)”), (C) a gellan gum (which may also be referred to as a “component (C)”), and (D) a water-soluble thickener (which may also be referred to as a “component (D)”).

### <Component (A)>

As the aliphatic alcohol having a melting point of 40° C. or higher, for example, a monohydric alcohol having an aliphatic hydrocarbon group having 13 to 22 carbon atoms can be used. The aliphatic hydrocarbon group may be linear, branched, saturated, or unsaturated. The component (A) may be used alone or in combination of two or more kinds thereof.

In the present specification, the melting point of an aliphatic alcohol refers to a numerical value measured by the following method.

About 5 mg of a sample is weighed and put in an aluminum sample pan. An aluminum cover is attached to this pan and the pan is installed in a differential scanning calorimeter “DSC7020” (manufactured by Hitachi High-Tech Science Corporation, product name). Using an electric cooling unit “Polyscience” (manufactured by Hitachi High-Tech Science Corporation, product name), under a nitrogen gas flow rate of 30 to 35 mL/min, the sample and a reference sample are held at −10° C. for 1 minute, then heated from 0° C. to 120° C. at a temperature rising rate of 10° C./min, cooled from 120° C. to −10° C. at a temperature lowering rate of −10° C./min, held at −10° C. for 5 minutes, and then heated again from −10° C. to 120° C. at a temperature rising rate of 10° C./min to obtain a melting endothermic curve. The peak temperature of the melting endothermic curve in the second temperature rise at this time is defined as the melting point.

Specific examples of the component (A) include lauryl alcohol, myristyl alcohol, cetyl alcohol, stearyl alcohol, behenyl alcohol, and oleyl alcohol. From the viewpoint of improving the shape retention of the cosmetic material and the usability of the cosmetic material, such as ease of spreading on the skin and good adhesiveness to the skin, in a well-balanced manner, the component (A) may be an alcohol having a linear saturated hydrocarbon group having 14 to 22 carbon atoms, or may be an alcohol having a linear saturated hydrocarbon group having 18 to 22 carbon atoms. Examples of the alcohol having a linear saturated hydrocarbon group having 14 to 17 carbon atoms include myristyl alcohol and cetyl alcohol, and examples of the alcohol having a linear saturated hydrocarbon group having 18 to 22 carbon atoms include stearyl alcohol and behenyl alcohol. As the component (A), these alcohols may be used in combination, or any one of these alcohols may be used.

From the viewpoint of improving the shape retention of the cosmetic material and the usability of the cosmetic material, such as ease of spreading on the skin and good adhesiveness to the skin, in a well-balanced manner, the content of the component (A) may be 1% to 20% by mass, may be 1% to 16% by mass, may be 1% to 12% by mass, may be 3% to 20% by mass, may be 3% to 16% by mass, may be 3% to 12% by mass, may be 6% to 20% by mass, may be 6% to 16% by mass, or may be 6% to 12% by mass with respect to the total amount of the cosmetic material. In addition, from the viewpoint of improving the shape retention of the cosmetic material while suppressing excessive application of the cosmetic material to the skin to improve the good adhesiveness, the content of the component (A) may be 1% by mass or more, 3% by mass or more, or 6% by mass or more; and from the viewpoint of improving the shape retention of the cosmetic material while suppressing the slippage of the cosmetic material on the skin to improve the good adhesiveness, the content of the component (A) may be 20% by mass or less, 16% by mass or less, or 12% by mass or less.

### <Other Oily Components>

The oil-in-water type solid cosmetic material of the present embodiment can contain oily components other than the component (A) (in which such oily components may be referred to as other oily components). Such other oily components may be a solid oil, a semi-solid oil, or a liquid oil. These can be used alone or in combination of two or more kinds thereof. Furthermore, in the present specification, the solid oil refers to an oil that is solid at 25° C., the semi-solid oil refers to an oil that is in a paste form at 25° C., and the liquid oil refers to an oil that is in a liquid form at 25° C.

The solid oil may be a plant-derived solid oil, may be an animal-derived solid oil, or may be a petroleum-derived solid oil or a synthetic solid oil. From the viewpoint of storage stability, the oil-in-water type solid cosmetic material of the present embodiment may include a plant-derived wax.

Examples of the plant-derived wax include waxes such as rice bran wax (rice wax), carnauba wax, candelilla wax, and sunflower seed wax; solid fats and oils such as hydrogenated castor oil, hydrogenated jojoba oil, palm oil, and Japan wax; and hydrocarbons such as candelilla wax hydrocarbon. Examples of the animal-derived solid oil include waxes such as beeswax, spermaceti, and lanolin; and esters such as cholesterol fatty acid ester. Examples of the other solid oils include hydrocarbons such as paraffin wax, microcrystalline wax, Fischer-Tropsch wax, polyethylene, ceresin, and ozokerite; esters such as glyceryl tribehenate; higher fatty acids such as stearic acid and palmitic acid; and silicones such as acrylic-modified silicone.

From the viewpoint of the moldability and the adhesiveness of the cosmetic material, the total content of the solid oils included in the cosmetic material (including the component (A)) may be 7% to 20% by mass, may be 7% to 16% by mass, may be 7% to 12% by mass, may be 8% to 20% by mass, may be 8% to 16% by mass, may be 8% to 12% by mass, may be 9% to 20% by mass, may be 9% to 16% by mass, or may be 9% to 12% by mass with respect to the total amount of the cosmetic material.

In the oil-in-water type solid cosmetic material of the present embodiment, the ratio (component (A)/solid oils) of the mass of the component (A) to the total mass of the solid oils (combined mass of solid oils in the component (A) and the other oily components) may be 0.1 to 1, may be 0.1 to 0.95, may be 0.1 to 0.9, may be 0.5 to 1, may be 0.5 to 0.95, may be 0.5 to 0.9, may be 0.7 to 1, or may be 0.7 to 0.95, and from the viewpoint of balancing the moldability, the shape retention, and the usability of the cosmetic material, and a pleasant sensory feel on the skin of the cosmetic material at a high level, the mass ratio may be 0.7 to 0.9.

Examples of the liquid oil include liquid oils and fats such as jojoba seed oil, sunflower seed oil, olive oil, castor oil, macadamia nut oil, camellia oil, rapeseed oil, linseed oil, and triglycerides; hydrocarbon oils such as liquid paraffin, squalane, mineral oil, and hydrogenated polyisobutene; ester oils such as diisostearyl malate, isopropyl myristate, cetyl 2-ethylhexanoate, isopropyl palmitate, ethylhexyl palmitate, glyceryl tri(caprylate/caprate), neopentyl glycol dicaprate, glyceryl tri(2-ethylhexanoate), polyglyceryl triisostearate, neopentyl glycol di(2-ethylhexanoate), and triethylhexanoin; higher alcohols such as oleyl alcohol, octyldodecanol, and isostearyl alcohol; higher fatty acids such as oleic acid, isostearic acid, linoleic acid, and linolenic acid; and silicone oils such as dimethylpolysiloxane (dimethicone), methylphenylpolysiloxane, octamethylcyclotetrasiloxane, decamethylcyclopentasiloxane, and methyl trimethicone.

Examples of the semi-solid oils include hydrocarbons such as petrolatum, and ester oils such as dipentaerythrityl hexa(hydroxystearate/stearate/rosinate), dipentaerythrityl tetra(hydroxystearate/isostearate), dipentaerythrityl penta(isostearate), dipentaerythrityl hexa(hydroxystearate), glyceryl tri(caprylate/caprate/myristate/stearate), hydrogenated castor oil isostearate, phytosteryl oleate, bis(behenyl/isostearyl/phytosteryl) dimer dilinoleyl dimer dilinoleate, bis(phytosteryl/isostearyl/cetyl/stearyl/behenyl) dimer dilinoleyl dimer dilinoleate, (phytosteryl/isostearyl/cetyl/stearyl/behenyl) dimer dilinoleate, bisstearyl dimer dilinoleyl dimer dilinoleate, and hydrogenated castor oil dimer dilinoleate.

### <Component (B)>

The component (B) is at least one kind of polyglycerin derivative surfactant selected from the group consisting of a polyglycerin fatty acid ester (B-1) (hereinafter also referred to as a component (B-1)) and a polyglycerin-modified silicone (B-2) (hereinafter also referred to as a component (B-2)). As the component (B), either one of the component (B-1) and the component (B-2) may be used, or these may be used in combination.

The HLB of the component (B) may be 8 or more and 17 or less, 8 or more and 15 or less, 8 or more and 12 or less, 8.5 or more and 17 or less, 8.5 or more and 15 or less, 8.5 or more and 12 or less, 9 or more and 17 or less, 9 or more and 15 or less, or 9 or more and 12 or less. In a case where the HLB value of the component (B) is within the range, the stability of the formulation is high. The HLB value can be determined based on the calculation formula described in “Emulsification and Solubilization Technology,” Kogaku Tosho Co., Ltd., published May 20, 1984, pp. 8-12.

Examples of the component (B-1) include components having polyglycerin to which 1 to 3 higher fatty acids having 8 to 22 carbon atoms are ester-bonded. The degree of polymerization of the polyglycerin is, for example, 4 to 10 or 6 to 10. Among these, the components in which two fatty acids are ester-bonded (polyglycerin diester) may be used. Examples of the polyglycerin diester include decaglyceryl distearate, decaglyceryl diisostearate, decaglyceryl dioleate, and decaglyceryl dimyristate. Among these, from the viewpoint of improving the shape retention of the cosmetic material and the usability of the cosmetic material, such as ease of spreading on the skin and good adhesiveness to the skin, in a well-balanced manner, decaglyceryl diisostearate (polyglyceryl-10 diisostearate) may be used as the component (B-1).

Examples of the component (B-2) include components in which polyglycerin is modified at a silicone chain in the main chain. In this case, the position at which the polyglycerin is introduced into the silicone chain in the main chain is optional. The component (B-2) may be of a branched type having polyglycerin at a side chain or a terminal of a silicone chain. Specific examples of the component (B-2) include polyglyceryl-3 polydimethylsiloxyethyl dimethicone, polyglyceryl-3 disiloxane dimethicone, lauryl polyglyceryl-3 polydimethylsiloxyethyl dimethicone, cetyl diglyceryl tris(trimethylsiloxy)silylethyl dimethicone, and bis(polyglyceryl-3-oxyphenylpropyl)dimethicone. Among these, from the viewpoint of improving the shape retention of the cosmetic material, the component (B-2) may be polyglyceryl-3 polydimethylsiloxyethyl dimethicone or polyglyceryl-3 disiloxane dimethicone.

From the viewpoint of improving the shape retention of the cosmetic material, the oil-in-water type solid cosmetic material may include at least one selected from the group consisting of polyglyceryl-10 diisostearate and polyglyceryl-3 disiloxane dimethicone as the component (B).

From the viewpoint of improving the moldability and the shape retention of the cosmetic material, the content of the component (B) with respect to the total amount of the cosmetic material may be 0.6% by mass or more, may be 0.75% by mass or more, or may be 10% by mass or more; from the viewpoint of improving the shape retention of the cosmetic material and the usability of the cosmetic material, such as ease of spreading on the skin and good adhesiveness to the skin, the content of the component (B) may be 5% by mass or less, may be 4% by mass or less, or may be 3% by mass or less; and from the above-described viewpoints, the content of the component (B) may be 0.6% to 5% by mass, may be 0.6% to 4% by mass, may be 0.6% to 3% by mass, may be 0.75% to 5% by mass, may be 0.75% to 4% by mass, may be 0.75% to 3% by mass, may be 1% to 5% by mass, may be 1% to 4% by mass, or may be 1% to 3% by mass.

### <Other Surfactants>

The oil-in-water type solid cosmetic material of the present embodiment can contain surfactants other than the component (B) (which may be referred to as “other surfactants”). Such other surfactants may be an anionic surfactant, a cationic surfactant, an amphoteric surfactant, or a nonionic surfactant. Such other surfactants can be used alone or in combination of two or more kinds thereof.

Examples of the nonionic surfactant include a polyoxyalkylene alkyl ether, a glycerin alkyl ether, a glycerin fatty acid ester, a sorbitan fatty acid ester, and alkylene glycol adducts thereof, a polyalkylene glycol fatty acid ester, and a polyether-modified silicone. Examples of the anionic surfactant include an alkyl phosphate, a polyoxyalkylene alkyl ether phosphate, a sulfonate, an alkyl sulfate, and a polyaspartate. Examples of the cationic surfactant include an alkylamine salt and an alkyltrimethylammonium salt. Examples of the amphoteric surfactant include hydrogenated lecithin, a carbobetaine-type amphoteric surfactant, a sulfobetaine-type amphoteric surfactant, and an amino acid-type amphoteric surfactant.

### <Component (C)>

The component (C) is gellan gum. The gellan gum is a type of water-soluble solidifying agent, which can produce a gel-like or solid-like mixture in a case of being mixed with water, and can solidify an oil-in-water type solid cosmetic material while imparting a moderate hardness to the cosmetic material. Furthermore, in the present specification, the gel-like or solid-like state refers to a state where the substance does not flow in a case of being tilted at room temperature (25° C.). As the gellan gum, gellan gum commercially available as a water-soluble solidifying agent for a cosmetic material can be used, and examples thereof include KELCOGEL HM, KELCOGEL DGA, KELCOGEL CG-HA, and KELCOGEL CG-LA. Among these, from the viewpoint of the moldability of the cosmetic material, deacylated gellan gum such as KELCOGEL CG-LA may be used.

From the viewpoint of sufficiently obtaining the moldability, the shape retention, and the usability of the cosmetic material, and a pleasant sensory feel on the skin of the cosmetic material, the content of the component (C) is 0.22% to 1.3% by mass with respect to the total amount of the cosmetic material. From the viewpoint of the shape retention of the cosmetic material, the content may be 0.24% by mass or more or may be 0.4% by mass or more; from the viewpoint of achieving both of ease of spreading on the skin and the adhesiveness, the content may be 1.10% by mass or less or may be 0.8% by mass or less; and from the above-described viewpoints, the content may be 0.22% to 1.1% by mass, may be 0.22% to 0.8% by mass, may be 0.24% to 1.3% by mass, may be 0.24% to 1.1% by mass, may be 0.24% to 0.8% by mass, may be 0.4% to 1.3% by mass, may be 0.4% to 1.10% by mass, or may be 0.4% to 0.8% by mass.

### <Component (D)>

As the water-soluble thickener, a known water-soluble thickener (however, excluding the component (C)) that is blended in a cosmetic material can be used. Examples of the water-soluble thickener include an acrylic acid amide compound, a polyacrylamide compound, a cellulose-based thickener, a polysaccharide-based thickener, and a viscosity-modifying mineral. The component (D) can be used alone or in combination of two or more kinds thereof. From the viewpoint of improving the moldability of the cosmetic material, the component (D) may include a polyacrylamide compound or a cellulose-based thickener, and may include the polyacrylamide compound among those.

Examples of the polyacrylamide compound include an acrylamide copolymer and a polyacrylamide. Among the polyacrylamide compounds, a polyacrylamide compound including an acryloyl dimethyltaurine salt as a constitutional unit may be used.

Examples of the polyacrylamide compound including the acryloyl dimethyltaurine salt as a constitutional unit include copolymers of hydroxyethyl acrylate and an acryloyl dimethyltaurine salt, such as a (hydroxyethyl acrylate/Na acryloyl dimethyltaurate) copolymer; copolymers of acrylate and acryloyl dimethyltaurine salt, such as an (Na acrylate/Na acryloyl dimethyltaurate) copolymer; an (ammonium acryloyl dimethyltaurate/VP) copolymer; and an (ammonium acryloyl dimethyltaurate beheneth-25 methacrylate) crosspolymer. Among these, the copolymers of hydroxyethyl acrylate and an acryloyl dimethyltaurine salt may be used, or the (hydroxyethyl acrylate/Na acryloyl dimethyltaurate) copolymer may be used.

As the component (D), a commercially available product containing a polyacrylamide compound can be used. Examples of a commercially available product containing a polyacrylamide include SEPIGEL 305 (a mixture of a polyacrylamide, hydrogenated polyisobutene (or C13-14 isoparaffin), laureth-7, and water; Seiwa Kasei Co., Ltd.); examples of a commercially available product containing a copolymer of hydroxyethyl acrylate and an acryloyl dimethyltaurine salt include SEPINOV EMT 10 (a (hydroxyethyl acrylate/Na acryloyl dimethyltaurate) copolymer; Seiwa Kasei Co., Ltd.), SIMULGEL NS (a mixture of a (hydroxyethyl acrylate/Na acryloyl dimethyltaurate) copolymer, squalane, polysorbate 60, and water; Seiwa Kasei Co., Ltd.), SIMULGEL FL (a mixture of a (hydroxyethyl acrylate/Na acryloyl dimethyltaurate) copolymer, isohexadecane, polysorbate 60, and water; Seiwa Kasei Co., Ltd.), and SEPIPLUS S (a mixture of a (hydroxyethyl acrylate/Na acryloyl dimethyltaurate) copolymer, polyisobutene, PEG-7 trimethylolpropane coconut ether, and water; Seiwa Kasei Co., Ltd.); examples of a commercially available product containing a copolymer of acrylate and an acryloyl dimethyltaurine salt include SIMULGEL EG QD (a mixture of an (Na acrylate/Na acryloyl dimethyltaurate) copolymer, isohexadecane, polysorbate 80, and water; Seiwa Kasei Co., Ltd.) and SEPILIFE NUDE ((a mixture of an Na acrylate/Na acryloyl dimethyltaurate) copolymer, a (C15-19) alkane, polyglyceryl-6 laurate, polyglycerin-6, and water; Seiwa Kasei Co., Ltd.); examples of a commercially available product including a copolymer of acrylamide and acrylate include SEPIPLUS 265 (a mixture of an (acrylamide/ammonium acrylate) copolymer, polyisobutene, polysorbate 20, and water; Seiwa Kasei Co., Ltd.); and examples of a commercially available product including a copolymer of acrylic acid, acrylamide, acrylate, and an acryloyl dimethyltaurine salt include SEPIPLUS 400 (a mixture of polyacrylate-13, polyisobutene, polysorbate 20, and water; Seiwa Kasei Co., Ltd.).

Examples of the cellulose-based thickener include cellulose derivatives such as methyl cellulose, ethyl cellulose, cellulose gum, hydroxyethyl cellulose, hydroxypropyl cellulose, and hydroxypropylmethyl cellulose.

Examples of the polysaccharide-based thickener include pectin, guar gum, xanthan gum, and gum arabic.

In a case where the component (D) contains a polyacrylamide compound or a cellulose-based thickener, the oil-in-water type solid cosmetic material may or may not include any of the water-soluble thickeners other than the polyacrylamide compound and the cellulose-based thickener.

From the viewpoint of improving the usability of the cosmetic material, such as ease of spreading on the skin and good adhesiveness to the skin, and the shape retention of the cosmetic material in a well-balanced manner, the content of the component (D) may be 0.1% to 1% by mass, may be 0.1% to 0.8% by mass, may be 0.1% to 0.7% by mass, may be 0.15% to 1% by mass, may be 0.15% to 0.8% by mass, may be 0.15% to 0.7% by mass, may be 0.2% to 1% by mass, may be 0.2% to 0.8% by mass, or may be 0.2% to 0.7% by mass with respect to the total amount of the cosmetic material.

In a case where the component (D) contains a polyacrylamide compound, from the viewpoint of improving the usability of the cosmetic material, such as ease of spreading on the skin and good adhesiveness to the skin, and the shape retention of the cosmetic material in a well-balanced manner, the content of the polyacrylamide compound may be 30% to 100% by mass, may be 50% to 100% by mass, or may be 80% to 100% by mass with respect to the total amount of the component (D). In addition, in this case, the content of the polyacrylamide compound may be 0.10% to 10% by mass, may be 0.10% to 0.8% by mass, may be 0.1% to 0.7% by mass, may be 0.15% to 1% by mass, may be 0.15% to 0.8% by mass, may be 0.15% to 0.7% by mass, may be 0.2% to 1% by mass, may be 0.2% to 0.8% by mass, or may be 0.2% to 0.7% by mass with respect to the total amount of the cosmetic material.

In a case where the component (D) contains a cellulose-based thickener, from the viewpoint of improving the usability of the cosmetic material, such as ease of spreading on the skin and good adhesiveness to the skin, and the shape retention of the cosmetic material in a well-balanced manner, the content of the cellulose-based thickener may be 50% to 100% by mass, may be 70% to 100% by mass, or may be 90% to 100% by mass with respect to the total amount of the component (D). In addition, in this case, the content of the cellulose-based thickener may be 0.1% to 1% by mass, may be 0.1% to 0.8% by mass, may be 0.1% to 0.7% by mass, may be 0.15% to 1% by mass, may be 0.15% to 0.8% by mass, may be 0.15% to 0.7% by mass, may be 0.2% to 1% by mass, may be 0.2% to 0.8% by mass, or may be 0.2% to 0.7% by mass with respect to the total amount of the cosmetic material.

In a case where the component (D) contains a polyacrylamide compound and a cellulose-based thickener, from the viewpoint of improving the usability of the cosmetic material, such as ease of spreading on the skin and good adhesiveness to the skin, and the shape retention of the cosmetic material in a well-balanced manner, the total content of the polyacrylamide compound and the cellulose-based thickener may be 50% to 100% by mass, may be 70% to 100% by mass, or may be 90% to 100% by mass with respect to the total amount of the component (D). In addition, in this case, the total content of the polyacrylamide compound and the cellulose-based thickener may be 0.1% to 1% by mass, may be 0.1% to 0.8% by mass, may be 0.1% to 0.7% by mass, may be 0.15% to 1% by mass, may be 0.15% to 0.8% by mass, may be 0.15% to 0.7% by mass, may be 0.2% to 1% by mass, may be 0.2% to 0.8% by mass, or may be 0.2% to 0.7% by mass with respect to the total amount of the cosmetic material.

In the oil-in-water type solid cosmetic material of the present embodiment, from the viewpoint of improving the usability of the cosmetic material, such as ease of spreading on the skin and good adhesiveness to the skin, and the shape retention of the cosmetic material in a well-balanced manner, the mass ratio C/D of the content of the component (C) to the content of the component (D) may be 0.3 to 5, may be 0.3 to 4.5, may be 0.3 to 3, may be 1 to 5, may be 1 to 4.5, may be 1 to 3, may be 2 to 5, may be 2 to 4.5, or may be 2 to 3.

In a case where the oil-in-water type solid cosmetic material of the present embodiment contains a polyacrylamide compound as the component (D), from the viewpoint of improving the usability of the cosmetic material, such as ease of spreading on the skin and good adhesiveness to the skin, and the shape retention of the cosmetic material in a well-balanced manner, the mass ratio of the component (C) to the polyacrylamide compound may be 0.3 to 5, may be 0.3 to 4.5, may be 0.3 to 3, may be 1 to 5, may be 1 to 4.5, may be 1 to 3, may be 2 to 5, may be 2 to 4.5, or may be 2 to 3.

In a case where the oil-in-water type solid cosmetic material of the present embodiment contains a cellulose-based thickener as the component (D), from the viewpoint of improving the usability of the cosmetic material, such as ease of spreading on the skin and good adhesiveness to the skin, and the shape retention of the cosmetic material in a well-balanced manner, the mass ratio of the component (C) to the cellulose-based thickener may be 0.3 to 5, may be 0.3 to 4.5, may be 0.3 to 3, may be 1 to 5, may be 1 to 4.5, may be 1 to 3, may be 2 to 5, may be 2 to 4.5, or may be 2 to 3.

In a case where the oil-in-water type solid cosmetic material of the present embodiment contains a polyacrylamide compound and a cellulose-based thickener as the component (D), from the viewpoint of improving the usability of the cosmetic material, such as ease of spreading on the skin and good adhesiveness to the skin, and the shape retention of the cosmetic material in a well-balanced manner, the mass ratio of the component (C) to the total mass of the polyacrylamide compound and the cellulose-based thickener may be 0.3 to 5, may be 0.3 to 4.5, may be 0.3 to 3, may be 1 to 5, may be 1 to 4.5, may be 1 to 3, may be 2 to 5, may be 2 to 4.5, or may be 2 to 3.

### <Other Water-Soluble Solidifying Agents>

The oil-in-water type solid cosmetic material of the present embodiment can contain water-soluble solidifying agents other than the gellan gum (which may be referred to as other water-soluble solidifying agents). Such other water-soluble solidifying agents only need to be a water-soluble solidifying agent that can produce a gel-like or solid-like mixture in a case of being mixed with water, and known water-soluble solidifying agents that are blended in cosmetic materials can be used. Examples of such other water-soluble solidifying agents include polysaccharides such as agar, carrageenan, and tamarind gum. The oil-in-water type solid cosmetic material may not include a water-soluble solidifying agent other than the gellan gum.

In a case where the oil-in-water type solid cosmetic material includes a water-soluble solidifying agent other than the gellan gum, from the viewpoint of achieving both the moldability and the sensory feel of the cosmetic material, the content of the water-soluble solidifying agent other than the gellan gum may be 0.01 to 10 parts by mass, may be 0.01 to 1 part by mass, or may be 0.01 to 0.1 parts by mass with respect to 100 parts by mass of the component (C).

From the viewpoint of the moldability and the shape retention of the cosmetic material, the total content of the water-soluble solidifying agents (including the component (C)) included in the oil-in-water type solid cosmetic material of the present embodiment may be 0.22% by mass or more, may be 0.24% by mass or more, or may be 0.4% by mass or more; and from the viewpoint of achieving both ease of spreading on the skin and the adhesiveness to the skin of the cosmetic material, the total content may be less than 2% by mass, may be 1% by mass or less, or may be 0.8% by mass or less with respect to the total amount of the cosmetic material. From the viewpoint of improving the usability such as ease of spreading on the skin and good adhesiveness to the skin, and the moldability and the shape retention in a well-balanced manner, the total content of the water-soluble solidifying agents (including the component (C)) included in the oil-in-water type solid cosmetic material of the present embodiment may be 0.22% by mass or more and less than 2% by mass, may be 0.22% by mass or more and 1% by mass or less, may be 0.22% by mass or more and 0.8% by mass or less, may be 0.24% by mass or more and less than 2% by mass, may be 0.24% by mass or more and 1% by mass or less, may be 0.24% by mass or more and 0.8% by mass or less, may be 0.4% by mass or more and less than 2% by mass, may be 0.4% by mass or more and 1% by mass or less, or may be 0.4% by mass or more and 0.8% by mass or less with respect to the total amount of the cosmetic material.

In the oil-in-water type solid cosmetic material of the present embodiment, from the viewpoint of improving the usability of the cosmetic material, such as ease of spreading on the skin and good adhesiveness to the skin, and the shape retention of the cosmetic material in a well-balanced manner, the mass ratio of the total content of the water-soluble solidifying agents (including the component (C)) included in the oil-in-water type solid cosmetic material to the content of the component (D) may be 0.3 to 5, may be 0.3 to 4.5, may be 0.3 to 3, may be 1 to 5, may be 1 to 4.5, may be 1 to 3, may be 2 to 5, may be 2 to 4.5, or may be 2 to 3.

In a case where the oil-in-water type solid cosmetic material of the present embodiment contains a polyacrylamide compound as the component (D), from the viewpoint of improving the usability of the cosmetic material, such as ease of spreading on the skin and good adhesiveness to the skin, and the shape retention of the cosmetic material in a well-balanced manner, the total content of the component (C) and the polyacrylamide compound may be 95 parts by mass or more, 99 parts by mass or more, 99.9 parts by mass or more, or 100 parts by mass with respect to 100 parts by mass of the total content of the water-soluble solidifying agents (including the component (C)) and the water-soluble thickener included in the cosmetic material.

In a case where the oil-in-water type solid cosmetic material of the present embodiment contains a cellulose-based thickener as the component (D), from the viewpoint of improving the usability of the cosmetic material, such as ease of spreading on the skin and good adhesiveness to the skin, and the shape retention of the cosmetic material in a well-balanced manner, the total content of the component (C) and the cellulose-based thickener may be 95 parts by mass or more, 99 parts by mass or more, 99.9 parts by mass or more, or 100 parts by mass with respect to 100 parts by mass of the total content of the water-soluble solidifying agents (including the component (C)) and the water-soluble thickener included in the cosmetic material.

In a case where the oil-in-water type solid cosmetic material of the present embodiment contains a polyacrylamide compound and a cellulose-based thickener as the component (D), from the viewpoint of improving the usability of the cosmetic material, such as ease of spreading on the skin and good adhesiveness to the skin, and the shape retention of the cosmetic material in a well-balanced manner, the total content of the component (C), the polyacrylamide compound, and the cellulose-based thickener may be 95 parts by mass or more, 99 parts by mass or more, 99.9 parts by mass or more, or 100 parts by mass with respect to 100 parts by mass of the total content of the water-soluble solidifying agents (including the component (C)) and the water-soluble thickener included in the cosmetic material.

### <Aqueous Solvent>

The oil-in-water type solid cosmetic material of the present embodiment can contain water as an aqueous solvent. From the viewpoint of balancing the moldability, the shape retention, and the usability of the cosmetic material, and the pleasant sensory feel on the skin, such as freshness and a cooling sensation, of the cosmetic material at a high level, the content of water may be 20% to 70% by mass, may be 20% to 60% by mass, may be 20% to 50% by mass, may be 30% to 70% by mass, may be 30% to 60% by mass, may be 30% to 50% by mass, may be 35% to 70% by mass, may be 35% to 60% by mass, or may be 35% to 50% by mass with respect to the total amount of the oil-in-water type solid cosmetic material. The oil-in-water type solid cosmetic material may further include a polyhydric alcohol such as 1,3-butylene glycol (BG) and glycerin as the aqueous solvent. From the viewpoint of the sensory feel such as freshness of the cosmetic material, the total content of the aqueous solvent may be 20% to 70% by mass, may be 20% to 60% by mass, may be 20% to 50% by mass, may be 30% to 70% by mass, may be 30% to 60% by mass, may be 30% to 50% by mass, may be 35% to 70% by mass, may be 35% to 60% by mass, or may be 35% to 50% by mass with respect to the total amount of the oil-in-water type solid cosmetic material.

### <Powder Component>

The oil-in-water type solid cosmetic material of the present embodiment can contain a powder component. The powder can be used without particular limitation as long as it is commonly used in a cosmetic product. Examples thereof include an extender pigment and a coloring pigment. As these powders, a commercially available product mixed with the above-described liquid oil in advance may be used. In addition, the oil-in-water type solid cosmetic material may not contain a powder component.

Examples of the extender pigment include inorganic powder, synthetic inorganic powder, organic powder, metal soap, and synthetic polymer powder. More specific examples thereof include glass powder, anhydrous silicic acid, aluminum silicate, magnesium silicate, aluminum magnesium silicate, mica, synthetic phlogopite (synthetic mica), kaolin, sericite, synthetic sericite, talc, phlogopite, synthetic mica, silica, calcium carbonate, magnesium carbonate, aluminum oxide, boron nitride, silicon carbide, barium sulfate, zinc stearate, aluminum stearate, zinc myristate, polyethylene powder, urethane beads, polymethyl methacrylate, and organopolysiloxane elastomers.

Examples of the coloring pigment include red iron oxide, yellow iron oxide, black iron oxide, cobalt oxide, chromium oxide, ultramarine, Prussian blue, titanium oxide, zinc oxide, fine particle titanium oxide, mica, and pearl pigments (mica titanium, iron oxide-coated mica titanium, fine particle titanium oxide-coated titanium mica, barium sulfate-coated titanium mica, fish scale foil, bismuth oxychloride, aluminum flakes, and the like). These powders may not be subjected to a surface treatment, or may be subjected to a surface treatment for the purpose of improving usability, dispersibility, and the like. Examples of the surface treatment include a metal soap, a silicone compound, a fluorine compound, a surfactant, and an amino acid compound.

From the viewpoint of improving the pleasant sensory feel on the skin, such as freshness and a cooling sensation, the usability such as ease of spreading on the skin and good adhesiveness to the skin, and the moldability and shape retention in a well-balanced manner, the content of the powder component may be 0% to 30% by mass, may be 0% to 20% by mass, may be 0% to 10% by mass, may be 0.1% to 30% by mass, may be 0.1% to 20% by mass, may be 0.1% to 10% by mass, may be 3% to 30% by mass, may be 3% to 20% by mass, or may be 3% to 10% by mass with respect to the total amount of the cosmetic material.

### <Other Components>

The oil-in-water type solid cosmetic material of the present embodiment can include components other than the above-described components. As such other components, components commonly used in cosmetic products, for example, aqueous components such as a moisturizer, an ultraviolet absorbing agent, an antifoaming agent, a preservative, vitamins, a beauty component, an antioxidant, a fragrance, and an emulsification stabilizer can be appropriately blended as necessary within a scope not impairing the effects of the present invention. The aqueous component can also be used as an aqueous solvent.

Examples of the moisturizer include polyhydric alcohols such as 1,3-butylene glycol (BG), dipropylene glycol (DPG), glycerin, 1,2-pentanediol, sorbitol, and mannitol. These can be used alone or in combination of two or more kinds thereof.

Examples of the emulsification stabilizer include inorganic salts such as sodium chloride, magnesium chloride, and magnesium sulfate. These can be used alone or in combination of two or more kinds thereof.

In the oil-in-water type solid cosmetic material of the present embodiment, from the viewpoint of satisfying the moldability, the shape retention, and the usability of the cosmetic material, and the pleasant sensory feel on the skin, such as freshness and a cooling sensation, of the cosmetic material at a high level, the total content of the components constituting the water phase may be 25% to 80% by mass, may be 25% to 75% by mass, may be 25% to 65% by mass, may be 30% to 80% by mass, may be 30% to 75% by mass, may be 30% to 65% by mass, may be 40% to 80% by mass, may be 40% to 75% by mass, or may be 40% to 65% by mass with respect to the total amount of the oil-in-water type solid cosmetic material.

In the oil-in-water type solid cosmetic material of the present embodiment, from the viewpoint of satisfying the moldability, the shape retention, and the usability of the cosmetic material, and the pleasant sensory feel on the skin, such as freshness and a cooling sensation, of the cosmetic material at a high level, the total content of the water, the component (C), and the component (D) may be 25% to 80% by mass, may be 25% to 75% by mass, may be 25% to 65% by mass, may be 30% to 80% by mass, may be 30% to 75% by mass, may be 30% to 65% by mass, may be 40% to 80% by mass, may be 40% to 75% by mass, or may be 40% to 65% by mass with respect to the total amount of the oil-in-water type solid cosmetic material.

From the viewpoint of improving the shape retention and the usability of the cosmetic material in a well-balanced manner, the hardness of the oil-in-water type solid cosmetic material of the present embodiment may be 0.7 to 1.5 N. Furthermore, in the present specification, the hardness of the cosmetic material means a value measured by the following procedure. First, a sample for measurement is prepared by filling a screw cup having a capacity of 30 mL with a cosmetic material that has been heated and melted at 85° C. or higher and allowing the cosmetic material to be left to stand overnight at 25° C. For this sample for measurement, a hardness is obtained by measuring the sample using FUDOH rheometer RT-2002D⋅D (manufactured by REO TEC Corporation) under the conditions of a pressure-sensitive shaft tip shape of a tubular form with a diameter of 3 mm, a penetration speed of 6 cm/min, a penetration depth of 10 mm, and a temperature of 25° C.

The oil-in-water type solid cosmetic material of the present embodiment may be three-dimensionally molded to be applied directly to the skin. The cosmetic material may have, for example, a stick shape or ajar shape. In the three-dimensionally molded oil-in-water type solid cosmetic material, the cosmetic material is held in a container main body such that at least a part of the cosmetic material protrudes from the container main body, and thus the cosmetic material can be directly applied to the skin. In this case, the cosmetic material may always protrude from the container main body, or may protrude from the container main body by an advancing mechanism of the container.

Examples of a use of the oil-in-water type solid cosmetic material of the present embodiment include makeup cosmetic materials such as a cheek color, an eyeshadow, a foundation, a lipstick, a concealer, a highlighter, a makeup base, and an eyebrow, skin care cosmetic materials such as a beauty serum stick and a moisturizer, body cosmetic materials such as a sunscreen and a solid perfume, and hair styling cosmetic materials such as a temporary hair dye.

### [Method for Producing Oil-In-Water Type Solid Cosmetic Material]

A method for producing the oil-in-water type solid cosmetic material of the present embodiment can be carried out, for example, by mixing the above-described component (A), component (B), component (C), component (D), and other components (powder components, other oily components, other aqueous components, and the like) in water, and filling a predetermined mold with the mixture to allow the mixture to be solidified.

As a procedure for mixing, for example, the oil phase including the component (A) and the other oil phase components and the water phase including the component (C), the component (D), the other aqueous components, and water may each be prepared, and the oil phase may be added to the water phase and stirred to be emulsified. The surfactant including the component (B) may be mixed with the oil phase, may be mixed with the water phase, or may be mixed with the oil phase and the water phase separately. In addition, the powder component may be mixed with the oil phase, may be mixed with the water phase, or may be mixed with the oil phase and the water phase separately.

The temperature at the time of preparing the oil phase may be a temperature at which the component (A) is dissolved in the solvent. The temperature at the time of preparing the water phase may be a temperature at which the component (C) and the component (D) are dissolved in the solvent.

As the predetermined mold, a mold may be used, or a general container used for a cosmetic product may be used. In a case where a mold is used as a mold for filling the cosmetic material, after the cosmetic material is solidified, the cosmetic material can be inserted into a holding body (a middle plate or the like) of a container for a cosmetic product and filled (insertion-type filling) to obtain a cosmetic product of an embodiment which will be described later. Since the oil-in-water type solid cosmetic material of the present embodiment can be molded into a desired shape and has sufficient shape retention, it may be molded by the above-described insertion-type filling.

A container that can be used as a mold generally has a tubular part (sleeve) that is a part of the container and a tubular holding body that is movable up and down inside the tubular part. Then, the inside of the tubular member can be directly filled with the mixture or the mixture that has been heated and melted to obtain a cosmetic product of an embodiment which will be described later. Since the oil-in-water type solid cosmetic material of the present embodiment can be molded into a desired shape and has sufficient shape retention, it may be molded by the above-described direct filling.

Examples of a method for the direct filling include a front filling method and a back filling method. In the front filling method, the opening part of the container for a cosmetic product is filled with the mixture. In this case, the opening part of the container for a cosmetic product may be covered with an upper mold (die) after the opening part is filled with a certain amount of the mixture. In the back filling method, the container for a cosmetic product is inverted and the mixture is filled through a filling hole provided in the bottom or the like of the container for a cosmetic product. In this case, the opening part of the container for a cosmetic product may be covered with an upper mold (die). Thereafter, in a case where the cosmetic material is cooled and solidified, the filling hole may be closed by a plug, a sealing material, or the like.

### [Cosmetic Product]

The cosmetic product of the present embodiment includes a container and the above-described oil-in-water type solid cosmetic material of the present embodiment stored in the container, in which the container has a container main body holding the oil-in-water type solid cosmetic material, the oil-in-water type solid cosmetic material being held in the container main body in a state of protruding from the container main body, or a container main body accommodating the oil-in-water type solid cosmetic material, and having a holding body capable of causing the oil-in-water type solid cosmetic material to protrude from the container main body while holding the oil-in-water type solid cosmetic material, and the oil-in-water type solid cosmetic material is applied by direct contact with skin.

Since the cosmetic product of the present embodiment includes the above-described oil-in-water type solid cosmetic material of the present embodiment, the cosmetic material can have a predetermined shape, has sufficient shape retention and usability such as ease of spreading on the skin and good adhesiveness to the skin, and can provide a pleasant sensory feel on the skin, such as freshness and a cooling sensation.

As the container, a general container used for a cosmetic product can be used, and for example, a general container used for lipsticks and the like, such as a container having a mechanism capable of allowing a stick-shaped cosmetic material to be advanced and retracted, can be used.

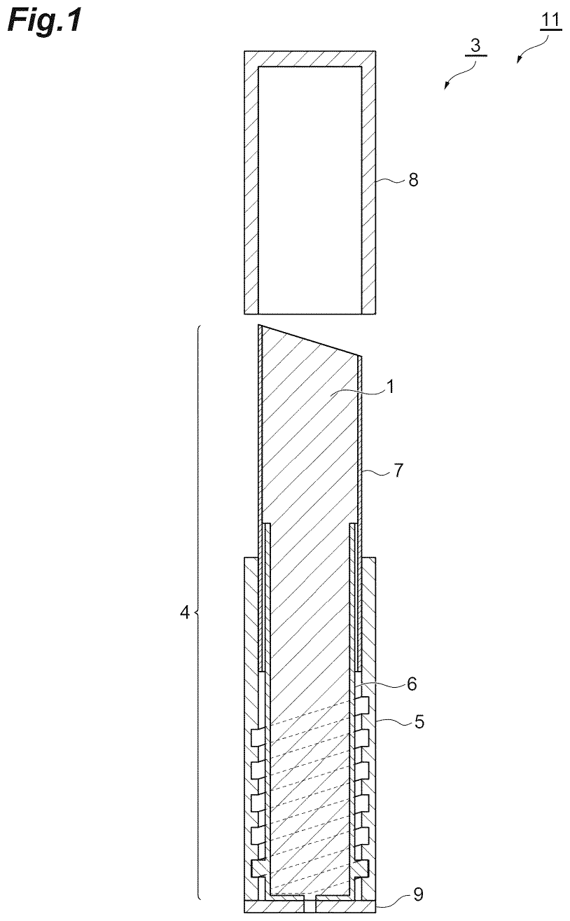

FIG. 1 is a schematic cross-sectional view showing an embodiment of a cosmetic product. A cosmetic product 11 shown in FIG. 1 includes a container 3 and an oil-in-water type solid cosmetic material 1 of the present embodiment accommodated in the container 3, and the container 3 includes a container main body 4, an upper lid 8 covering an upper part of the container main body 4, and a bottom lid 9 covering a lower part of the container main body 4. The container main body 4 includes a holding body 6 holding the oil-in-water type solid cosmetic material 1, and a mechanism allowing the holding body 6 to be advanced and retracted. In the container main body 4, the holding body 6 can be advanced and retracted by a shaft 5 having a spiral groove inside and holding the holding body 6 by the groove and a sleeve 7 transmitting a movement in a rotation direction to the holding body 6. In the cosmetic product 11, the holding body 6 and the bottom lid 9 have a filling hole at the center of the bottom part, and the filling hole can be filled with a mixture of raw materials constituting the oil-in-water type solid cosmetic material 1 by a back filling method, so that the oil-in-water type solid cosmetic material 1 can be molded.

### EXAMPLES

Hereinafter, the present invention will be described in more detail with reference to Examples, but the technical scope of the present invention is not limited to these Examples.

### [Preparation of Oil-In-Water Type Solid Cosmetic Material]

### Examples 1 to 11 and Comparative Examples 1 to 9

An oil-in-water type solid cosmetic material (cheek color) was prepared by the following production method with a formulation shown in Tables 1 to 3 (the numerical value of a blending amount indicates the content (% by mass) based on the total amount of the cosmetic material).

### <Production Method>

Each component of the water phase group was mixed with purified water, heated to 85° C. or higher, and then uniformly dispersed to form a water phase after confirming that the water-soluble solidifying agent was completely dissolved. On the other hand, the components of the oil phase group were mixed, heated to 85° C. or higher, dissolved, and homogenized to obtain an oil phase. Subsequently, the oil phase was gradually added to the water phase while stirring the water phase to emulsify the oil phase, thereby obtaining a mixed solution. The mixed solution was filled in a mold of a stick-shaped container having an advancing mechanism in a state where the mixed solution was kept at 60° C. or higher, and then left to be cooled at room temperature for 1 hour or longer, thereby obtaining an oil-in-water type solid cosmetic material in a state where the upper surface was smooth.

The obtained oil-in-water type solid cosmetic material was evaluated for each item based on the following evaluation methods. The results are shown in Tables 1 to 3.

### <Moldability of Cosmetic Material>

The state of the cosmetic material after cooling in the production method was evaluated according to the following evaluation standards. Furthermore, in Comparative Examples 2 and 3, the cosmetic material remained liquid even after cooling, and was not solidified.

### [Evaluation Standards]

OK: Molded into the shape of the mold
NG: Not molded into the shape of the mold (the cosmetic material is solidified but is brittle; is not solidified; is gel-like; and the like)

### <Evaluation of Cosmetic Material>

Five panels specialized in evaluation of cosmetic products were directly asked to apply the cosmetic materials of Examples and Comparative Examples so that the upper surface of the cosmetic material was in contact with the inner side of the arm, and were asked to evaluate the cosmetic material from the viewpoint of ease of spreading, good adhesiveness (coloring efficiency), and a sensory feel such as freshness and a cooling sensation. Furthermore, each evaluation was performed on a five-point scale according to the following evaluation standards, with each sample being given a score. Furthermore, the average score of all panels was determined according to the following determination standards.

### [Score: Evaluation Standards]

5 points: Very good
4 points: Good
3 points: Normal
2 points: Slightly defective
1 point: Defective

### [Determination Standards (Average Score of Scores)]

A: 4 or more
B: 3 or more and less than 4
C: 2 or more and less than 3
D: Less than 2

### <Shape Retention>

Five panels specialized in evaluation of cosmetic products were directly asked to apply the cosmetic materials of Examples and Comparative Examples so that the upper surface of the cosmetic material was in contact with the inner side of the arm, and were asked to evaluate the state of the cosmetic materials after 10 reciprocations of the application. Furthermore, each evaluation was performed on a five-point scale according to the following evaluation standards, with each sample being given a score. Furthermore, the average score of all panels was determined according to the following determination standards.

### [Score: Evaluation Standards]

5 points: The cosmetic material is not chipped.
4 points: An edge of the cosmetic material is slightly chipped.
3 points: An edge of the cosmetic material is chipped overall.
2 points: The cosmetic material is broken overall.
1 point: The cosmetic material detaches.

### [Determination Standards (Average Score of Scores)]

A: 4 or more
B: 3 or more and less than 4
C: 2 or more and less than 3
D: Less than 2

TABLE 1

| | | | Exam- | Exam- | Exam- | Exam- | Exam- | Exam- | Exam- |
|---|---|---|---|---|---|---|---|---|---|
| | | | ple 1 | ple 2 | ple 3 | ple 4 | ple 5 | ple 6 | ple 7 |
| Water | Solvent | Water | 43.23 | 43.23 | 43.23 | 43.23 | 43.23 | 42.74 | 43.23 |
| phase | | BG | 5.50 | 5.50 | 5.50 | 5.50 | 5.50 | 5.50 | 5.50 |
| | Component (C) | Gellan gum | 0.60 | 0.60 | 0.60 | 0.60 | 0.60 | 1.00 | 0.60 |
| | Component (D) | Polyacrylamide compound 1 | 0.25 | 0.25 | 0.25 | 0.25 | 0.25 | 0.34 | 0.25 |
| | Glycerin | | 2.50 | 2.50 | 2.50 | 2.50 | 2.50 | 2.50 | 2.50 |
| | Phenoxyethanol | | 0.50 | 0.50 | 0.50 | 0.50 | 0.50 | 0.50 | 0.50 |
| | Sodium chloride | | 0.20 | 0.20 | 0.20 | 0.20 | 0.20 | 0.20 | 0.20 |
| Oil phase | Component (A) | Behenyl alcohol | 7.00 | 10.00 | 15.00 | 10.00 | 10.00 | 10.00 | 10.00 |
| | Other oily | Squalane | 7.00 | 7.00 | 7.00 | 7.00 | 7.00 | 7.00 | 7.00 |
| | components | Dimethicone | 7.00 | 7.00 | 7.00 | 7.00 | 7.00 | 7.00 | 7.00 |
| | | Methyl trimethicone | 9.60 | 6.50 | 1.50 | 7.50 | 4.50 | 6.50 | 6.50 |
| | | Neopentyl glycol diethylhexanoate | 3.00 | 3.00 | 3.00 | 3.00 | 3.00 | 3.00 | 3.00 |
| | | Carnauba wax | 1.70 | 1.70 | 1.70 | 1.70 | 1.70 | 1.70 | 1.70 |
| | | Trimethylsiloxysilicate | 2.50 | 2.50 | 2.50 | 2.50 | 2.50 | 2.50 | 2.50 |
| | | (Dimethicone/vinyl dimethicone) | 2.00 | 2.00 | 2.00 | 2.00 | 2.00 | 2.00 | 2.00 |
| | | crosspolymer | | | | | | | |
| | Component (B) | Polyglycerin-modified silicone | 2.00 | 2.00 | 2.00 | 1.00 | 4.00 | 2.00 | — |
| | | Polyglycerin fatty acid ester 1 | — | — | — | — | — | — | 2.00 |
| | Other surfactants | PEG-60 hydrogenated castor oil | 1.00 | 1.00 | 1.00 | 1.00 | 1.00 | 1.00 | 1.00 |
| | Powder component | Dimethicone-treated spherical silica | 0.50 | 0.50 | 0.50 | 0.50 | 0.50 | 0.50 | 0.50 |
| | | (particle diameter: 5 μm) | | | | | | | |
| | | Alkylsilane-treated porous silica | 2.50 | 2.50 | 2.50 | 2.50 | 2.50 | 2.50 | 2.50 |
| | | (particle diameter: 2 to 7 μm) | | | | | | | |
| | | Dimethicone-treated mica-based pearl | 1.00 | 1.00 | 1.00 | 1.00 | 1.00 | 1.00 | 1.00 |
| | | Iron oxide | 0.50 | 0.50 | 0.50 | 0.50 | 0.50 | 0.50 | 0.50 |

| | Antioxidant | 0.02 | 0.02 | 0.02 | 0.02 | 0.02 | 0.02 | 0.02 |
|---|---|---|---|---|---|---|---|---|
| Total | | 100 | 100 | 100 | 100 | 100 | 100 | 100 |
| Evaluation | Moldability | OK | OK | OK | OK | OK | OK | OK |
| results | Ease of spreading | A | A | C | A | B | B | A |
| | Good adhesiveness (coloring efficiency) | B | A | C | A | C | B | A |
| | Shape retention | A | A | A | B | B | A | B |
| | Sensory feel | A | A | C | A | C | B | A |

TABLE 2

| | | | | | | | Compar- | Compar- | Compar- |
|---|---|---|---|---|---|---|---|---|---|
| | | | Example | Example | Example | Example | ative | ative | ative |
| | | | 8 | 9 | 10 | 11 | Example 1 | Example 2 | Example 3 |
| Water | Solvent | Water | 43.08 | 43.23 | 43.23 | 43.23 | 43.23 | 43.23 | 43.23 |
| phase | | BG | 5.50 | 5.50 | 5.50 | 5.50 | 5.50 | 5.50 | 5.50 |
| | Component (C) | Gellan gum | 0.60 | 0.60 | 0.60 | 0.60 | 0.60 | 0.60 | 0.60 |
| | Component (D) | Polyacrylamide compound 1 | — | — | 0.25 | 0.25 | 0.25 | 0.25 | 0.25 |
| | | Polyacrylamide compound 2 | 0.40 | — | — | — | — | — | — |
| | | Cellulose gum | — | 0.25 | — | — | — | — | — |
| | Other | Polysorbate 80 | — | — | — | — | — | — | 2.00 |
| | surfactants | | | | | | | | |
| | Glycerin | | 2.50 | 2.50 | 2.50 | 2.50 | 2.50 | 2.50 | 2.50 |
| | Phenoxyethanol | | 0.50 | 0.50 | 0.50 | 0.50 | 0.50 | 0.30 | 0.50 |
| | Sodium chloride | | 0.20 | 0.20 | 0.20 | — | 0.20 | 0.20 | 0.20 |
| | Magnesium sulfate | | — | — | — | 0.20 | — | — | — |
| Oil phase | Component (A) | Behenyl alcohol | 10.00 | 10.00 | — | 10.00 | — | 10.00 | 10.00 |
| | | Stearyl alcohol | ? | — | 10.00 | — | — | — | — |
| | Other oily | Squalane | 7.00 | 7.00 | 7.00 | 7.00 | 7.00 | 7.00 | 7.00 |
| | components | Dimethicone | 7.00 | 7.00 | 7.00 | 7.00 | 7.00 | 7.00 | 7.00 |
| | | Methyl trimethicone | 6.50 | 6.50 | 6.50 | 6.50 | 6.50 | 6.50 | 6.50 |
| | | Neopentyl glycol diethylhexanonte | 3.00 | 3.00 | 3.00 | 3.00 | 3.00 | 3.00 | 3.00 |
| | | Carnauba wax | 1.70 | 1.70 | 1.70 | 1.70 | 11.70 | 1.70 | 1.70 |
| | | Trimethylsiloxysilicate | 2.50 | 2.50 | 2.50 | 2.50 | 2.50 | 2.50 | 2.50 |
| | | (Dimethicone/vinyl dimethicone) | 2.00 | 2.00 | 2.00 | 2.00 | 2.00 | 2.00 | 2.00 |
| | | crosspolymer | | | | | | | |
| | Component (B) | Polyglycerin-modified silicone | 2.00 | 2.00 | 2.00 | 2.00 | 2.00 | — | — |
| | Other | Polyether-modified silicone | — | — | — | — | — | 2.00 | — |
| | surfactants | PEG-60 hydrogenated castor oil | 1.00 | 1.00 | 1.00 | 1.00 | 1.00 | 1.00 | 1.00 |
| | Powder | Dimethicone-treated spherical | 0.50 | 0.50 | 0.50 | 0.50 | 0.50 | 0.50 | 0.50 |
| | component | silica (particle diameter: 5 μm) | | | | | | | |
| | | Alkylsilane-treated porous silica | 2.50 | 2.50 | 2.50 | 2.50 | 2.50 | 2.50 | 2.50 |
| | | (particle diameter: 2 to 7 μm) | | | | | | | |
| | | Dimethicone-treated mica-based pearl | 1.00 | 1.00 | 1.00 | 1.00 | 1.00 | 1.00 | 1.00 |
| | | Iron oxide | 0.50 | 0.50 | 0.50 | 0.50 | 0.50 | 0.50 | 0.50 |

| | Antioxidant | 0.02 | 0.02 | 0.02 | 0.02 | 0.02 | 0.02 | 0.02 |
|---|---|---|---|---|---|---|---|---|
| Total | | 100 | 100 | 1.00 | 100 | 100 | 100 | 100 |
| Evaluation | Moldability | OK | OK | OK | OK | OK | NG | NG |
| results | Base of spreading | B | B | A | A | B | D | D |
| | Good adhesiveness (coloring efficiency) | B | C | A | A | D | D | D |
| | Shape retention | B | A | C | A | A | D | D |
| | Sensory feel | B | C | A | A | D | D | D |

TABLE 3

| | | | Compar- | Compar- | Compar- | Compar- | Compar- | Compar- |
|---|---|---|---|---|---|---|---|---|
| | | | ative | ative | ative | ative | ative | ative |
| | | | Example 4 | Example 5 | Example 6 | Example 7 | Example 8 | Example 9 |
| Water | Solvent | Water | 43.23 | 41.83 | 42.83 | 42.83 | 43.63 | 42.33 |
| phase | | BG | 5.50 | 5.50 | 5.50 | 5.50 | 5.50 | 5.50 |
| | Component (C) | Gellan gum | — | — | — | — | 0.20 | 1.50 |
| | Agar | | 0.60 | 2.00 | — | — | — | — |
| | Tamarind gum | | — | — | 1.00 | — | — | — |
| | Carrageenan | | — | — | — | 1.00 | — | — |
| | Component (D) | Polyacrylamide compound 1 | 0.25 | 0.25 | 0.25 | 0.25 | 0.25 | 0.25 |
| | Glycerin | | 2.50 | 2.50 | 2.50 | 2.50 | 2.50 | 2.50 |
| | Phenoxyethanol | | 0.50 | 0.50 | 0.50 | 0.50 | 0.50 | 0.50 |
| | Sodium chloride | | 0.20 | 0.20 | 0.20 | 0.20 | 0.20 | 0.20 |
| Oil phase | Component (A) | Behenyl alcohol | 10.00 | 7.00 | 10.00 | 10.00 | 10.00 | 10.00 |
| | Other oily | Squalane | 7.00 | 7.00 | 7.00 | 7.00 | 7.00 | 7.00 |
| | components | Dimethicone | 7.00 | 7.00 | 7.00 | 7.00 | 7.00 | 7.00 |
| | | Methyl trimethicone | 6.50 | 9.50 | 6.50 | 6.50 | 6.50 | 6.50 |
| | | Neopentyl glycol diethylhexanoate | 3.00 | 3.00 | 3.00 | 3.00 | 3.00 | 3.00 |
| | | Carnauba wax | 1.70 | 1.70 | 1.70 | 1.70 | 1.70 | 1.70 |
| | | Trimethylsiloxysilicate | 2.50 | 2.50 | 2.50 | 2.50 | 2.50 | 2.50 |
| | | (Dimethicone/vinyl dimethicone) | 2.00 | 2.00 | 2.00 | 2.00 | 2.00 | 2.00 |
| | | crosspolymer | | | | | | |
| | Component (B) | Polvelycerin-modified silicone | 2.00 | 2.00 | 2.00 | 2.00 | 2.00 | 2.00 |
| | Other | PEG-60 hydrogenated castor oil | 1.00 | 1.00 | 1.00 | 1.00 | 1.00 | 1.00 |
| | surfactants | | | | | | | |
| | Powder | Dimethicone-treated spherical silica | 0.50 | 0.50 | 0.50 | 0.50 | 0.50 | 0.50 |
| | component | (particle diameter: 5 μm) | | | | | | |
| | | Alkylslune-treated porous silica (article | 2.50 | 2.50 | 2.50 | 2.50 | 2.50 | 2.50 |
| | | diameter: 2 to 7 μm) | | | | | | |
| | | Dimethicone-treated mica-based pearl | 1.00 | 1.00 | 1.00 | 1.00 | 1.00 | 1.00 |
| | | Iron oxide | 0.50 | 0.50 | 0.50 | 0.50 | 0.50 | 0.30 |

| | Antioxidant | 0.02 | 0.02 | 0.02 | 0.02 | 0.02 | 0.02 |
|---|---|---|---|---|---|---|---|
| Total | | 100 | 100 | 100 | 100 | 100 | 100 |
| Evaluation | Moldability | OK | OK | NG | NG | OK | OK |
| results | Ease of spreading | D | D | D | D | B | D |
| | Good adhesiveness (coloring efficienes) | C | D | D | D | B | D |
| | Shape retention | D | C | D | D | D | C |
| | Sensory fool | D | D | D | D | B | D |

In Tables 1 to 3, the details of each component are as follows.

### <Component (A)>

Behenyl alcohol: Solid oil, melting point 66° C.
Stearyl alcohol: Solid oil, melting point 61° C.

### <Other Oily Components>

Dimethicone: Liquid oil, viscosity of 6 mPa·s

The melting point of the solid oil was measured according to the above-described measuring method. In addition, the viscosity of the liquid oil was measured according to the following measuring method.

### (Method for Measuring Viscosity)

The viscosity of the sample at 25° C. was measured using a Brookfield type viscometer (BM type) under the following conditions. Furthermore, the measurement time was set to 1 minute.
5 to 50 mPa·s: BM type, BL adapter, rotation speed of 12 rpm
50 to 500 mPa·s: BM type, rotor No. 1, rotation speed: 12 rpm
250 to 2,500 mPa·s: BM type, rotor No. 2, rotation speed: 12 rpm
1,000 to 10,000 mPa·s: BM type, rotor No. 3, rotation speed: 12 rpm
5,000 to 50,000 mPa·s: BM type, rotor No. 4, rotation speed: 12 rpm

### <Component (B)>

Polyglycerin-modified silicone: Polyglyceryl-3 disiloxane dimethicone (manufactured by Shin-Etsu Chemical Co., Ltd., product name “KF-6100”, HLB: medium)
Polyglycerin fatty acid ester 1: Polyglyceryl-10 diisostearate (HLB 11.1)

### <Other Surfactants>

Polyether-modified silicone: PEG-11 methyl ether dimethicone (manufactured by Shin-Etsu Chemical Co., Ltd., product name “KF-6011”, HLB 14.5)
PEG-60 hydrogenated castor oil: Polyethylene glycol ether of hydrogenated castor oil (average number of moles added: 60, HLB 14)
Polysorbate 80: Nonionic surfactant (HLB 15)

### <Component (C)>

Gellan gum: (manufactured by Iwase Cosfa Co., Ltd., product name “KELCOGEL CG-LA”)

### <Component (D)>

Polyacrylamide compound 1: (Hydroxyethyl acrylate/Na acryloyl dimethyltaurate) copolymer mixture (manufactured by SEPPIC Inc., product name “SEPINOV EMT 10”)
Polyacrylamide compound 2: Polyacrylamide mixture (manufactured by SEPPIC Inc., product name “SEPIGEL 305”, component concentration: 40%)

### Example 12: Cheek Color

| | |
|---|---|
| | (Formulation proportion |
| (Component) | (% by mass)) |

| | | |
|---|---|---|
| 1. | Water | 45.28 |
| 2. | BG | 6.00 |
| 3. | Glycerin | 1.50 |
| 4. | Gellan gum | 0.25 |
| 5. | Phenoxyethanol | 0.50 |
| 6. | Polyacrylamide compound 1 | 0.80 |
| 7. | Magnesium chloride | 0.20 |
| 8. | Behenyl alcohol | 6.50 |
| 9. | Squalane | 8.00 |
| 10. | Dimethicone | 3.80 |
| 11. | Methyl trimethicone | 2.00 |
| 12. | Carnauba wax | 2.50 |
| 13. | Trimethylsiloxysilicate | 3.00 |
| 14. | Lauroyl lysine | 2.50 |
| 15. | (Dimethicone/vinyl dimethicone) | 1.20 |
| | crosspolymer | |
| 16. | PEG-60 hydrogenated castor oil | 1.00 |
| 17. | Mica titanium | 2.50 |
| 18. | Titanium oxide | 0.75 |
| 19. | Synthetic fluorphlogopite | 9.00 |
| 20. | Iron oxide | 0.50 |
| 21. | Red 226 | 0.20 |
| 22. | Polyglycerin-modified silicone | 2.00 |
| 23. | Antioxidant | 0.02 |

| | |
|---|---|
| Total | 100.00 |

The details of the components are the same as those described above.

### <Production Method>

An oil-in-water type solid cosmetic material was obtained in the same manner as in Example 1, except that the components 2 to 7 were mixed with purified water (component 1) as components of the water phase group and the components 8 to 23 were mixed as components of the oil phase group.

### <Evaluation>

As a result of performing the same evaluations as described above on the obtained oil-in-water type solid cosmetic materials, it was confirmed that the moldability was “OK”, the ease of spreading was “B”, the good adhesiveness (coloring efficiency) was “B”, the shape retention was “B”, and the sensory feel was “B”.

### Example 13: Cheek Color

| | |
|---|---|
| | (Formulation proportion |
| (Component) | (% by mass)) |

| | | |
|---|---|---|
| 1. | Water | 43.98 |
| 2. | BG | 6.00 |
| 3. | Glycerin | 1.50 |
| 4. | Gellan gum | 0.25 |
| 5. | Phenoxyethanol | 0.50 |
| 6. | Polyacrylamide compound 1 | 0.80 |
| 7. | Behenyl alcohol | 9.00 |
| 8. | Squalane | 7.00 |
| 9. | Dimethicone | 3.80 |
| 10. | Methyl trimethicone | 1.20 |
| 11. | Neopentyl glycol diethylhexanoate | 3.00 |
| 12. | Carnauba wax | 2.00 |
| 13. | Trimethylsiloxysilicate | 1.80 |
| 14. | Lauroyl lysine | 2.50 |
| 15. | (Dimethicone/vinyl dimethicone) | 1.20 |
| | crosspolymer | |
| 16. | PEG-60 hydrogenated castor oil | 1.00 |
| 17. | Mica titanium | 2.50 |
| 18. | Titanium oxide | 0.75 |
| 19. | Synthetic fluorphlogopite | 1.00 |
| 20. | Mica | 7.50 |
| 21. | Iron oxide | 0.50 |
| 22. | Red 226 | 0.20 |
| 23. | Polyglycerin fatty acid ester 1 | 2.00 |
| 24. | Antioxidant | 0.02 |

| | |
|---|---|
| Total | 100.00 |

The details of the components are the same as those described above.

### <Production Method>

An oil-in-water type solid cosmetic material was obtained in the same manner as in Example 1, except that the components 2 to 6 were mixed with purified water (component 1) as components of the water phase group and the components 7 to 24 were mixed as components of the oil phase group.

### <Evaluation>

As a result of performing the same evaluations as described above on the obtained oil-in-water type solid cosmetic materials, it was confirmed that the moldability was “OK”, the ease of spreading was “B”, the good adhesiveness (coloring efficiency) was “B”, the shape retention was “B”, and the sensory feel was “B”.

### Example 14: Cheek Color

| | |
|---|---|
| | (Formulation proportion |
| (Component) | (% by mass)) |

| | | |
|---|---|---|
| 1. | Water | 45.98 |
| 2. | BG | 6.00 |
| 3. | Glycerin | 1.50 |
| 4. | Gellan gum | 0.25 |
| 5. | Phenoxyethanol | 0.50 |
| 6. | Polyacrylamide compound 1 | 0.80 |
| 7. | Behenyl alcohol | 9.00 |
| 8. | Squalane | 10.00 |
| 9. | Dimethicone | 3.80 |
| 10. | Methyl trimethicone | 1.20 |
| 11. | Lauroyl lysine | 2.50 |
| 12. | Trimethylsiloxysilicate | 1.80 |
| 13. | (Dimethicone/vinyl dimethicone) | 1.20 |
| | crosspolymer | |
| 14. | PEG-60 hydrogenated castor oil | 1.00 |
| 15. | Synthetic fluorphlogopite | 1.00 |
| 16. | Mica | 7.50 |
| 17. | Mica titanium | 2.50 |
| 18. | Titanium oxide | 0.75 |
| 19. | Iron oxide | 0.50 |
| 20. | Red 226 | 0.20 |
| 21. | Polyglycerin fatty acid ester 2 | 2.00 |
| 22. | Antioxidant | 0.02 |

| | |
|---|---|
| Total | 100.00 |

The details of the components are the same as those described above except for the components shown below.
Polyglycerin fatty acid ester 2: Polyglyceryl-10 dioleate (HLB 11)

### <Production Method>

An oil-in-water type solid cosmetic material was obtained in the same manner as in Example 1, except that the components 2 to 6 were mixed with purified water (component 1) as components of the water phase group and the components 7 to 22 were mixed as components of the oil phase group.

### <Evaluation>

As a result of performing the same evaluations as described above on the obtained oil-in-water type solid cosmetic materials, it was confirmed that the moldability was “OK”, the ease of spreading was “B”, the good adhesiveness (coloring efficiency) was “B”, the shape retention was “C”, and the sensory feel was “B”.

### Example 15: Cheek Color

| | |
|---|---|
| | (Formulation proportion |
| (Component) | (% by mass)) |

| | | |
|---|---|---|
| 1. | Water | 45.98 |
| 2. | BG | 6.00 |
| 3. | Glycerin | 1.50 |
| 4. | Gellan gum | 0.25 |
| 5. | Phenoxyethanol | 0.50 |
| 6. | Polyacrylamide compound 1 | 0.80 |
| 7. | Behenyl alcohol | 9.00 |
| 8. | Squalane | 10.00 |
| 9. | Dimethicone | 3.80 |
| 10. | Methyl trimethicone | 1.20 |
| 11. | Lauroyl lysine | 2.50 |
| 12. | Trimethylsiloxysilicate | 1.80 |
| 13. | (Dimethicone/vinyl dimethicone) | 1.20 |
| | crosspolymer | |
| 14. | PEG-60 hydrogenated castor oil | 1.00 |
| 15. | Synthetic fluorphlogopite | 1.00 |
| 16. | Mica | 7.50 |
| 17. | Mica titanium | 2.50 |
| 18. | Titanium oxide | 0.75 |
| 19. | Iron oxide | 0.50 |
| 20. | Red 226 | 0.20 |
| 21. | Polyglycerin fatty acid ester 3 | 2.00 |
| 22. | Antioxidant | 0.02 |

| | |
|---|---|
| Total | 100.00 |

The details of the components are the same as those described above except for the components shown below.
Polyglycerin fatty acid ester 3: Polyglyceryl-10 dimyristate (HLB 12.3)

### <Production Method>

An oil-in-water type solid cosmetic material was obtained in the same manner as in Example 1, except that the components 2 to 6 were mixed with purified water (component 1) as components of the water phase group and the components 7 to 22 were mixed as components of the oil phase group.

### <Evaluation>

As a result of performing the same evaluations as described above on the obtained oil-in-water type solid cosmetic materials, it was confirmed that the moldability was “OK”, the ease of spreading was “B”, the good adhesiveness (coloring efficiency) was “B”, the shape retention was “C”, and the sensory feel was “B”.

### Example 16: Beauty Serum Stick

| | |
|---|---|
| | (Formulation proportion |
| (Component) | (% by mass)) |

| | | |
|---|---|---|
| 1. | Water | Balance |
| 2. | BG | 10.00 |
| 3. | Glycerin | 7.00 |
| 4. | Gellan gum | 0.60 |
| 5. | Phenoxyethanol | 0.50 |
| 6. | Polyacrylamide compound 1 | 0.25 |
| 7. | Sodium chloride | 0.20 |
| 8. | Sodium hyaluronate | 0.015 |
| 9. | Lavender flower extract | 0.007 |
| 10. | Behenyl alcohol | 10.00 |
| 11. | Squalane | 7.00 |
| 12. | Dimethicone | 7.00 |
| 13. | Methyl trimethicone | 5.00 |
| 14. | Jojoba seed oil | 3.50 |
| 15. | Neopentyl glycol diethylhexanoate | 3.00 |
| 16. | (Dimethicone/vinyl dimethicone) | 2.00 |
| | crosspolymer | |
| 17. | Polyglycerin-modified silicone | 2.00 |
| 18. | Carnauba wax | 1.70 |
| 19. | PEG-60 hydrogenated castor oil | 1.00 |
| 20. | Antioxidant | 0.02 |

| | |
|---|---|
| Total | 100.00 |

The details of the components are the same as those described above.

### <Production Method>

A beauty serum stick was obtained in the same manner as in Example 1, except that the components 2 to 9 were mixed with purified water (component 1) as components of the water phase group and the components 10 to 20 were mixed as components of the oil phase group.

### <Evaluation>

The same evaluations as described above were performed on the obtained beauty serum sticks, except that the good adhesiveness to the skin was evaluated from the viewpoint of the usability instead of the coloring efficiency, and it was confirmed that the moldability was evaluated as “OK”, the ease of spreading was evaluated as “A”, the good adhesiveness was evaluated as “A”, the shape retention was evaluated as “A”, and the sensory feel was evaluated as “A”.

### Example 17: Stick Eyeshadow

| | | (Formulation proportion |
|---|---|---|
| (Component) | | (% by mass)) |
| 1. | Water | 26.69 |
| 2. | BG | 6.00 |
| 3. | Glycerin | 3.50 |
| 4. | Gellan gum | 0.60 |
| 5. | Phenoxyethanol | 0.50 |
| 6. | Polyacrylamide compound 1 | 0.25 |
| 7. | Alkyl acrylate derivative | 7.00 |
| | emulsion aqueous dispersion | |
| | film-forming agent | |
| 8. | Sodium chloride | 0.20 |
| 9. | Behenyl alcohol | 10.00 |
| 10. | Squalane | 7.00 |
| 11. | Dimethicone | 5.50 |
| 12. | Methyl trimethicone | 4.00 |
| 13. | Neopentyl glycol diethylhexanoate | 3.00 |
| 14. | Jojoba seed oil | 3.00 |
| 15. | Polyglycerin-modified silicone | 2.00 |
| 16. | Carnauba wax | 1.70 |
| 17. | (Dimethicone/vinyl dimethicone) | 1.70 |
| | crosspolymer | |
| 18. | PEG-60 hydrogenated castor oil | 1.00 |
| 19. | Dimethicone-treated spherical silica | 0.50 |
| | (particle diameter: 5 μm) | |
| 20. | Dimethicone-treated mica | 1.50 |
| 21. | Dimethicone-treated mica titanium | 11.50 |
| 22. | Dimethicone-treated mica black | 1.80 |
| 23. | Iron oxide | 1.00 |
| 24. | Red 226 | 0.04 |
| 25. | Antioxidant | 0.02 |

| | |
|---|---|
| Total | 100.00 |

The details of the components are the same as those described above except for the components shown below.
Alkyl acrylate derivative emulsion aqueous dispersion film-forming agent: (solid content: 30%, water: 70%)

### <Production Method>

A stick eyeshadow was obtained in the same manner as in Example 1, except that the components 2 to 8 were mixed with purified water (component 1) as components of the water phase group and the components 9 to 25 were mixed as components of the oil phase group.

### <Evaluation>

The same evaluations as described above were performed on the obtained stick eyeshadows, and it was confirmed that the moldability was evaluated as “OK”, the ease of spreading was evaluated as “A”, the good adhesiveness (coloring efficiency) was evaluated as “A”, the shape retention was “A”, and the sensory feel was evaluated as “A”.

### <Measurement of Hardness>

The hardness of the cosmetic materials of Examples 2, 12, 13, 16, and 17 was measured by the following procedure.

First, the cosmetic material was heated to 85° C. or higher to be melted and filled in a screw cup having a capacity of 30 mL, and was then left to stand at 25° C. overnight to manufacture a sample for measurement. For this sample for measurement, the hardness was measured using FUDOH rheometer RT-2002D⋅D (manufactured by REO TEC Corporation) under the conditions of a pressure-sensitive shaft tip shape of a tubular form with a diameter of 3 mm, a penetration speed of 6 cm/min, a penetration depth of 10 mm, and a temperature of 25° C. The results are shown in Table 4.

TABLE 4

| | Exam- | Exam- | Exam- | Exam- | Exam- |
|---|---|---|---|---|---|
| | ple 2 | ple 12 | ple 13 | ple 16 | ple 17 |
| Hardness | 0.84 | 0.81 | 1.26 | 1.43 | 1.10 |
| [N] | | | | | |

### REFERENCE SIGNS LIST

1 . . . oil-in-water type solid cosmetic material
3 . . . container
4 . . . container main body
5 . . . shaft
6 . . . holding body
7 . . . sleeve
8 . . . upper lid
9 . . . bottom lid
11 . . . cosmetic product.

What is claimed is:

1. An oil-in-water type solid cosmetic material comprising:
(A) an aliphatic alcohol having a melting point of 40° C. or higher;
(B) at least one kind of polyglycerin derivative surfactant selected from the group consisting of a polyglycerin fatty acid ester and a polyglycerin-modified silicone;
(C) a gellan gum; and
(D) a water-soluble thickener,
wherein a content of the component (C) is 0.22% to 1.3% by mass with respect to a total amount of the oil-in-water type solid cosmetic material.

2. The oil-in-water type solid cosmetic material according to claim 1,
wherein a total content of water-soluble solidifying agents (including the component (C)) included in the oil-in-water type solid cosmetic material is 1% by mass or less with respect to the total amount of the oil-in-water type solid cosmetic material.

3. The oil-in-water type solid cosmetic material according to claim 1,
wherein a total content of solid oils included in the oil-in-water type solid cosmetic material is 7% to 20% by mass with respect to the total amount of the oil-in-water type solid cosmetic material.

4. The oil-in-water type solid cosmetic material according to claim 1,
wherein a mass ratio C/D of the content of the component (C) to a content of the component (D) is 0.3 to 5.

5. The oil-in-water type solid cosmetic material according to claim 1,
wherein the component (D) includes a polyacrylamide compound including an acryloyl dimethyltaurine salt as a constitutional unit.

6. The oil-in-water type solid cosmetic material according to claim 1, further comprising:
a plant-derived wax.

7. The oil-in-water type solid cosmetic material according to claim 1,
wherein a content of water in the oil-in-water type solid cosmetic material is 20% to 70% by mass with respect to the total amount of the oil-in-water type solid cosmetic material.

8. The oil-in-water type solid cosmetic material according to claim 1,
wherein the oil-in-water type solid cosmetic material has a hardness of 0.7 to 1.5 N.

9. The oil-in-water type solid cosmetic material according to claim 1,
wherein the component (A) is an alcohol having a linear saturated hydrocarbon group having 18 to 22 carbon atoms.

10. The oil-in-water type solid cosmetic material according to claim 1,
wherein the oil-in-water type solid cosmetic material includes at least one selected from the group consisting of polyglyceryl-10 diisostearate and polyglyceryl-3 disiloxane dimethicone as the component (B).

11. The oil-in-water type solid cosmetic material according to claim 1,
wherein a total content of solid oils included in the oil-in-water type solid cosmetic material is 7% to 20% by mass with respect to the total amount of the oil-in-water type solid cosmetic material,
a content of the component (A) in the oil-in-water type solid cosmetic material is 6% to 12% by mass with respect to the total amount of the oil-in-water type solid cosmetic material,
a content of the component (B) in the oil-in-water type solid cosmetic material is 1% to 3% by mass with respect to the total amount of the oil-in-water type solid cosmetic material,
a mass ratio C/D of the content of the component (C) to a content of the component (D) is 0.3 to 5, and
a content of water in the oil-in-water type solid cosmetic material is 20% to 70% by mass with respect to the total amount of the oil-in-water type solid cosmetic material.

12. A cosmetic product comprising:
a container; and
the oil-in-water type solid cosmetic material according to claim 1 stored in the container,
wherein the container has
a container main body holding the oil-in-water type solid cosmetic material, the oil-in-water type solid cosmetic material being held in the container main body in a state of protruding from the container main body, or
a container main body accommodating the oil-in-water type solid cosmetic material, and having a holding body capable of causing the oil-in-water type solid cosmetic material to protrude from the container main body while holding the oil-in-water type solid cosmetic material, and
the oil-in-water type solid cosmetic material is applied by direct contact with skin.

13. The cosmetic product according to claim 12,
wherein the oil-in-water type solid cosmetic material has a stick shape.
